(19) United States
(12) Patent Application Publication
(10) Pub. No.: US 2016/0008625 A1
(43) Pub. Date: Jan. 14, 2016

(54) MEDICAL APPARATUS, SYSTEM AND METHOD

(71) Applicant: POLYPHOTONIX LIMITED, Durham (GB)

(72) Inventors: Luke Stuart Barclay, Gateshead (GB); Duncan John Hill, Durham (GB); Richard Anthony Kirk, London (GB); Thomas Snell, Stockton-on-Tees (GB); Melanie Jayne Winter, Peterlee (GB)

(73) Assignee: Polyphotonix Limited, Sedgefield, Durham (GB)

(21) Appl. No.: 14/765,796

(22) PCT Filed: Feb. 3, 2014
(86) PCT No.: PCT/GB2014/050289

§ 371 Date: Aug. 4, 2015

(30) Foreign Application Priority Data
Feb. 4, 2013 (GB) 1301958.3

(51) Int. Cl. A61N 5/06

(52) U.S. Cl. CPC A61N 5/0613; A61N 2005/0626; A61N 2005/0648; A61N 2005/0653

(57) ABSTRACT

A medical apparatus, system and method of producing a medical apparatus are disclosed. The apparatus includes a radiation source for emitting electromagnetic radiation towards an area to be treated of a patient; a mount element arranged to be worn by the patient for positioning the radiation source in a predetermined position relative to the area to be treated; and a controller for controlling the duration or time that the radiation source emits electromagnetic radiation, and for varying the intensity of electromagnetic radiation emitted in accordance with predetermined parameters.

The present invention relates to a medical apparatus, system and method. In particular, but not exclusively, the present invention relates to a medical apparatus, for example a facial mask, bandage or plaster, including a radiation source for treating a patient, which is controllable to specific patients' needs.

Phototherapy has been used for various therapeutic and cosmetic purposes. It generally involves the use of specific wavelengths of light radiation being administered to a patient. Phototherapy may be used to treat chronic infections such as hepatitis (A, B or C), bacterial infections, wounds, precancer conditions, seasonal affective disorder (SAD), various dermatological and cosmetic purposes such as skin rejuvenation, and various eye diseases such as diabetic macular edema, retinopathy of prematurity, wet or dry age-related macular degeneration and diabetic retinopathy, for example.

Diabetic retinopathy is a condition in which damage to the retina in the eye occurs and is caused by diabetes. More specifically, diabetic retinopathy is the result of microvascular retinal changes where hyperglycemia-induced intramural pericyte death and thickening of the basement membrane cause damage to the wall of blood vessels in the eye. This damage changes the formation of the blood-retinal barrier and also makes the retinal blood vessels become more permeable. Small blood vessels, such as those in the eye, are particularly vulnerable to poor blood sugar control. An overaccumulation of glucose and/or fructose damages the blood vessels in the retina. Damaged blood vessels are likely to leak fluid and lipids onto the macula. This condition can therefore lead to impaired vision and ultimately blindness. The condition can be treated by preventing the complete dark adaptation of the eye by providing some degree of light radiation to the eyes or eyelids during sleep. This is because, during dark adaptation, the eye requires an increased oxygen level, and thus the blood vessels must work harder during dark adaptation. Therefore by preventing complete dark adaptation of the eye, the blood vessels are less stressed and can rejuvenate over time. For diabetic retinopathy, preferably light having a wavelength of between around 460 to 550 nm is administered to the eyes or eyelids, which corresponds to the scotopic sensitivity of the eye. Of course for other diseases or conditions, other wavelength ranges may be useful.

It has been found useful to administer the radiation to the eye area by providing a mask type of device for a patient to wear during sleep, the mask configured to be secured over the patient's head to cover the eye area, and adapted to include light emitting sources in the region of the eyes. The light sources may be electroluminescent emitters, light emitting devices, light emitting cells (LECs), light emitting electrochemical cells (LEECs), LEDs or OLEDs, for example, and are arranged to emit light towards the eye area. The radiation acts to stimulate the rods of the eye leading to hyperpolarization and desensitization of the rod cells, which lowers their metabolic rates and hence results in a drop in oxygen consumption in the retina.

There are 3 known types of photoreceptor cells in the eye. Rods, cones and photosensitive retinal ganglion cells (pRGC), of which the cones can be further subdivided according to the particular opsin they contain (long (r), medium (g) and short (b) wavelength). Rods and cones are responsible for vision, and each type responds to a particular range of wavelengths, with rods being substantially more sensitive to low light levels than cones, but cones being better adapted to brighter light. Vision in low light levels where the rods are the dominant photoreceptor is known as Scotopic vision (10⁻⁶-10⁻² cd/m²), and the range of vision in which cones are primarily active is known as Photopic vision (1-10⁶ cd/m²). The borderline between the two is referred to as Mesopic vision (10⁻²-1 cd/m²). Colour is perceived by comparison between the response rates of different cell types. pRGCs are not involved in vision but are thought to be important in sleep cycles, melatonin generation and pupillary response.

WO2011/135362 discloses a radiation treatment apparatus for directing electromagnetic radiation into a patient's eyes. Radiation treatment may be started or stopped by a patient input (on/off switch) to switch at least one organic semiconductor radiation emitting device on or off. Here, the or each organic semiconductor radiation emitting device comprises an organic light emitting diode (OLED). Advantageously, the heat output from an OLED is less that that generated by a conventional light emitting diode (LED). OLEDs also emit light over a larger surface area than conventional LEDs, which assists in ensuring that radiation is directed correctly through the patient's eyelids and pupil to reach the retina of the eye. The or each OLED is mounted in a mask, goggles or a visor so that the electromagnetic radiation emitted by the or each OLED is directed into at least one eye of the patient, with the or each OLED in a predetermined position relative to the or each eye of the patient. Preferably, the mask, goggles or visor are provided with a securing strap or other means for securing the or each OLED to the patients face or head.

The radiation treatment apparatus disclosed WO2011/135362 may include a power supply and a controller for controlling the supply of power to the OLEDs. This provides the flexibility to vary the time and intensity of radiation exposure as part of a treatment regime. The duration and conditions of operation of the OLEDs may be recorded in a memory.

It would be useful to increase the usability and operability of a medical apparatus to provide increased functionality to users and clinicians alike.

According to a first aspect of the present invention there is provided a medical apparatus comprising:
a radiation source for emitting electromagnetic radiation towards an area to be treated of a patient;
a mount element arranged to be worn by the patient for positioning the radiation source in a predetermined position relative to the area to be treated; and
a controller for controlling the duration or time that the radiation source emits electromagnetic radiation, and for varying the intensity, waveform, frequency or pulse modulation of electromagnetic radiation emitted in accordance with predetermined parameters.

According to a second aspect of the present invention there is provided a system comprising:
a patient data monitoring apparatus for taking readings of a parameter associated with a patient; and
a treatment apparatus for being worn by the patient, comprising:
   a radiation source for emitting electromagnetic radiation towards an area to be treated of a patient;
   a mount element for positioning the radiation source in a predetermined position relative to the area to be treated; and
   a controller for controlling the intensity, waveform, frequency or pulse modulation of electromagnetic radiation emitted,
wherein the treatment apparatus is arranged to receive patient data, indicative of the readings or a proportion of the readings, from the patient data monitoring apparatus, and
   wherein the controller is arranged to vary the intensity, waveform, frequency or pulse modulation of the electromagnetic radiation emitted in accordance with the patient data.

According to a third aspect of the present invention there is provided a system comprising:
a treatment apparatus for being worn by a patient, comprising
   a radiation source for emitting electromagnetic radiation towards an area to be treated of a patient, and
   a mount element for positioning the radiation source in a predetermined position relative to the area to be treated; and
a controller for generating signals to control and vary the intensity, waveform, frequency or pulse modulation of electromagnetic radiation emitted by the radiation source, in accordance with predetermined parameters.

According to a fourth aspect of the present invention there is provided a method of manufacturing a medical apparatus comprising:
providing a radiation source for emitting electromagnetic radiation;
providing a mount element to be worn by a patient for positioning the radiation source in a predetermined position; and
providing a controller for controlling the duration or time that the radiation source emits electromagnetic radiation, and varying the intensity, waveform, frequency or pulse modulation of electromagnetic radiation emitted in accordance with predetermined parameter.

Certain embodiments of the invention provide the advantage that the intensity and/or optionally also the wavelength, waveform, frequency or pulse modulation of electromagnetic radiation used to treat an area of a patient may be varied in accordance with predetermined parameters, for example parameters associated with the patient themselves, or parameters associated with generalisations of the type of patient being treated. The parameters may include temperature, blood pressure level, the patient's age gender or race, the patient's response to test radiation levels, for example.

Embodiments of the invention are further described hereinafter with reference to the accompanying drawings, in which:

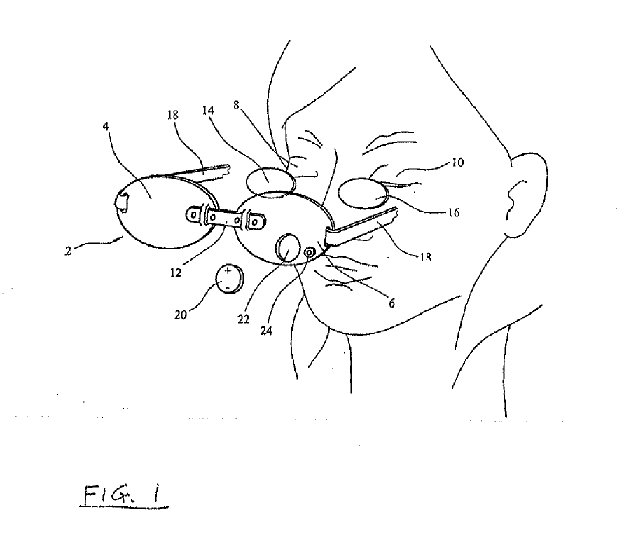

FIG. 1 illustrates an exploded perspective view of a radiation treatment apparatus;

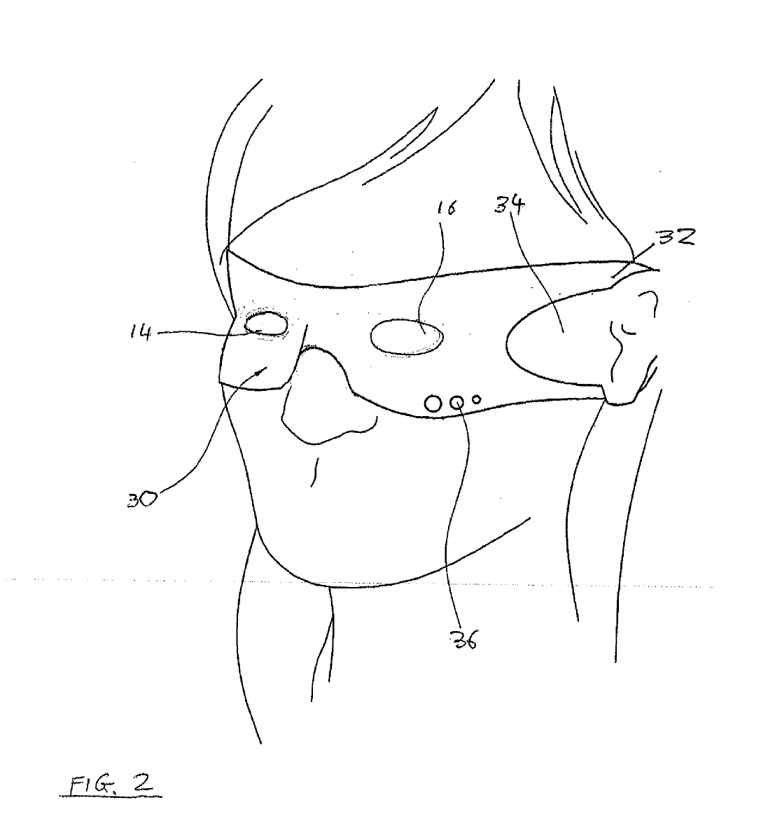

FIG. 2 illustrates an alternative radiation treatment apparatus;

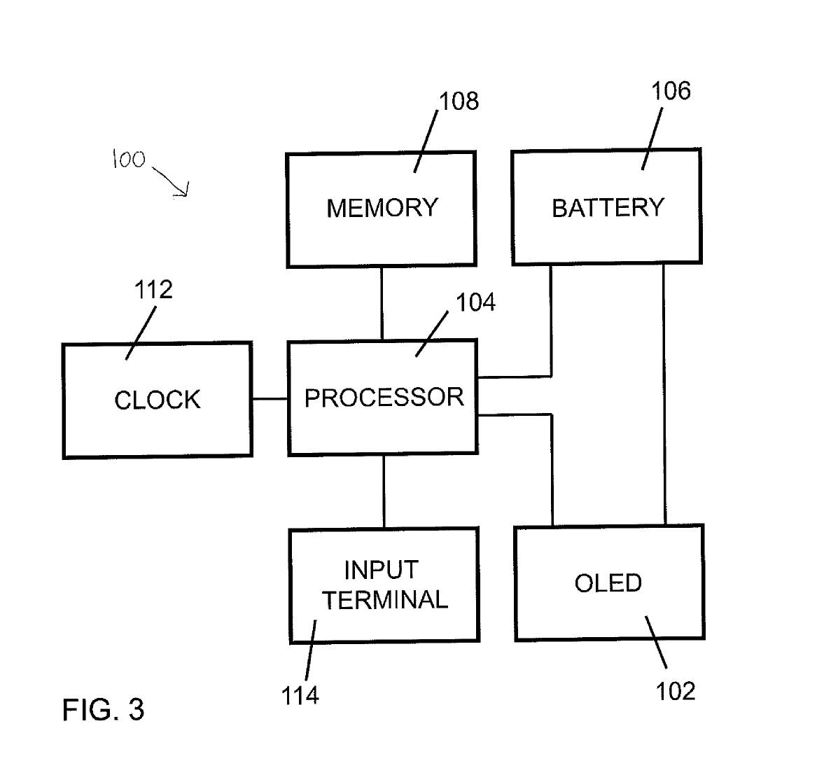

FIG. 3 schematically illustrates a radiation treatment apparatus according to an embodiment of the present invention;

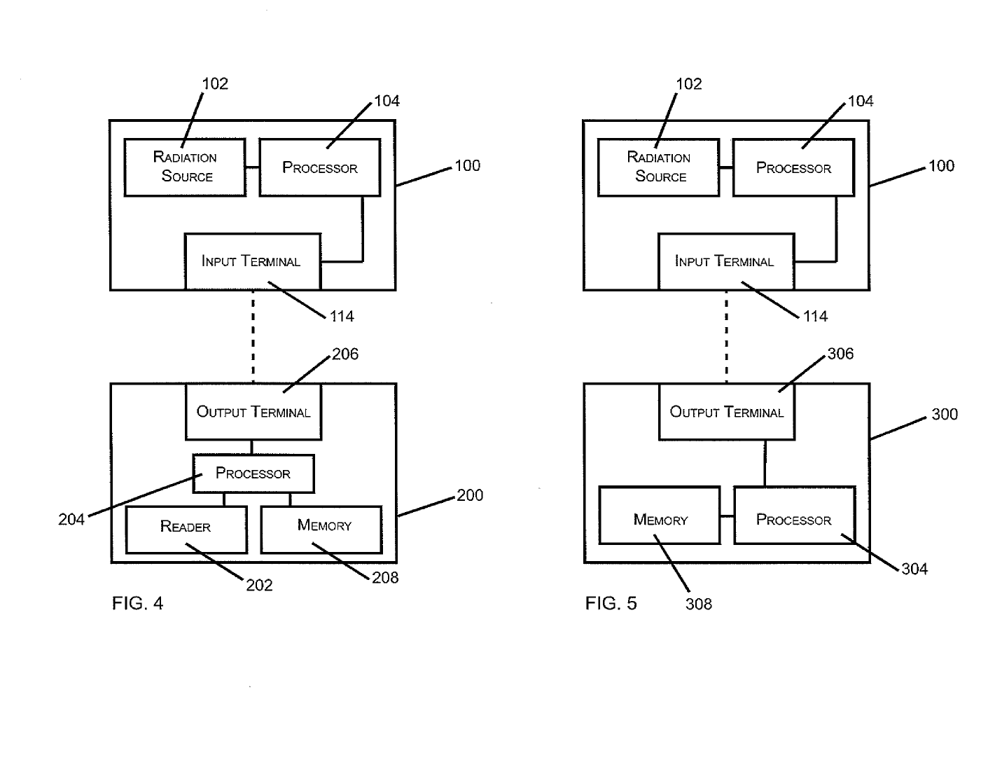

FIG. 4 illustrates a system including a radiation treatment apparatus and a patient monitoring device;

FIG. 5 illustrates a system including a radiation treatment apparatus and an external controller;

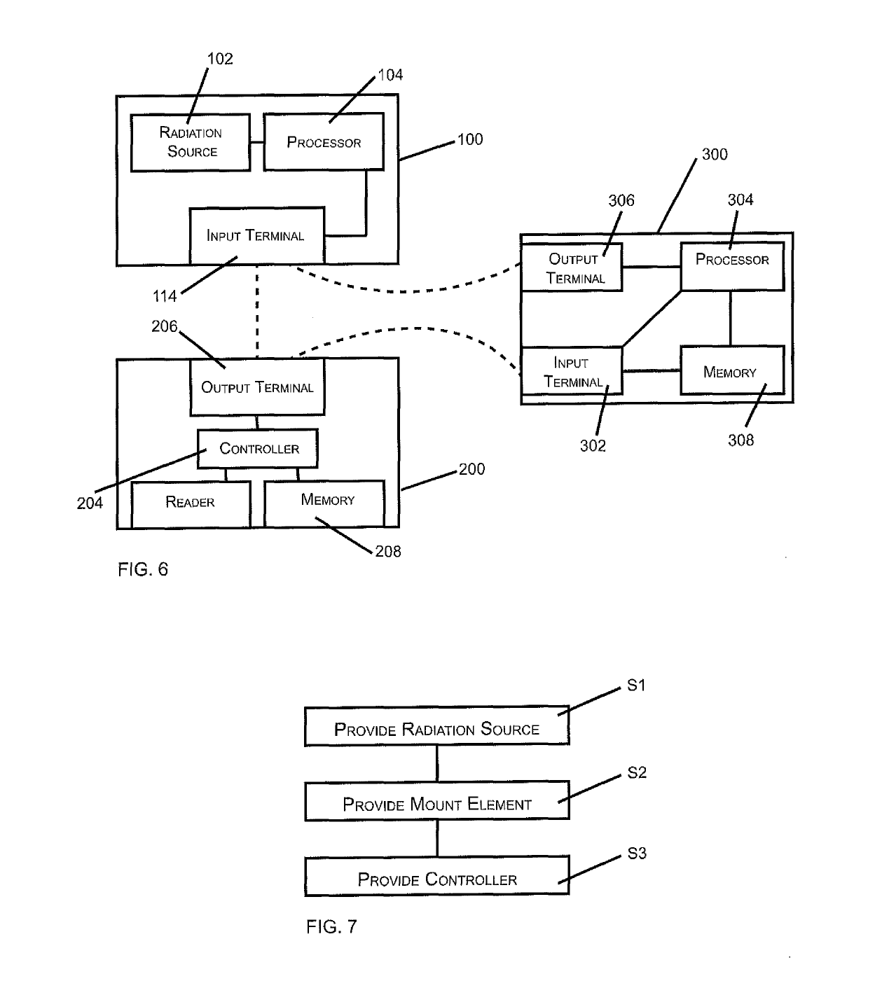

FIG. 6 illustrates a system including a radiation treatment apparatus, external controller and patient monitoring device; and

FIG. 7 shows a flow chart of a method of providing a radiation treatment apparatus.

In the drawings like reference numerals refer to like parts.

Certain elements of the apparatus described in WO2011/135362 may be used with the present invention. The contents of WO2011/135362 are incorporated herein by reference.

As illustrated in FIG. 1, an exploded perspective view of a radiation treatment apparatus 2 as disclosed in WO2011/135362 comprises supports (or mounts) 4, 6 to be located adjacent to the eyes of a patient, the supports 4, 6 each supporting a respective OLED 14, 16. It will be appreciated that other radiation emitting devices could be used, though OLEDs are particularly advantageous for the reasons given above. It has been found that OLEDs emitting radiation within the range 460 nm to 550 nm, centred at 480 nm to 550 nm, are particularly suitable for treatment of diabetic retinopathy. This is because when the radiation is filtered through the eyelids 8, 10 of a patient who is asleep, radiation centred at about 498 to 510 nm reaches the retinas of the patient, which is particularly efficacious for the treatment of diabetic retinopathy or wet AMD. Alternatively, radiation centred at about 670 nm may be useful for the treatment of dry AMD, for example. Of course other ranges of wavelengths or light radiation are known to be useful to treat other conditions. It will also be appreciated that the dosage regime for light radiation will also likely include the time period for which radiation treatment occurs, the frequency of the periods, and luminance of the light radiation (measured by candela per metre squared—cd/m²). Other conditions will of course require different dosage regimes. An adjustable strap 12 couples the supports 4, 6 together so that the spacing between the OLEDs 14, 16 can be matched to the spacing between a patient's eyes. A securing strap 18 secures the apparatus to the patients head. The OLEDs 14, 16 are powered by at least one battery 20 housed in at least one recess 22 and activated by a switch 24.

WO2011/135362 discloses a range of alternative embodiments, for instance mounting the OLEDs in a face mask. The face mask may be formed from a flexible material. The face mask may be secured by a strap similar to strap 18 shown in FIG. 1, or it may, for instance, be adhesively mounted to the patient's eye socket or face. Alternatively, the OLEDs could be integrated into a visor mounted to the patient's head via a head strap. FIG. 2 illustrates an alternative radiation treatment apparatus disclosed in WO2011/135362 that takes the form of a mask. The mask comprises a flexible portion 30 to conform to the shape of the patient's face. The flexible portion 30 extends to form straps 32 which extend either around the patient's head or are secured by the patient's ears passing through apertures 34. The OLEDs 14, 16 are incorporated into the flexible mask such that they are brought into close proximity to the patient's eyes. FIG. 2 further illustrates one or more sensors 36, for instance to sense ambient light levels, body temperature or movement of the patient for use in controlling the operation of the apparatus to minimise disturbance to the user's sleep.

The present invention provides a radiation treatment apparatus comprising an improvement to the radiation treatment apparatuses disclosed in WO2011/135362.

Referring now to FIG. 3, this schematically illustrates components of an apparatus 100 in accordance with an embodiment of the present invention. It will be appreciated that the purpose of FIG. 3 is to present the main functional units of an apparatus in accordance with an embodiment of the present invention, and places no limitation on the actual structure of the apparatus. However, one such structure would be a structure as depicted in FIG. 1 or FIG. 2, for example. The apparatus 100 comprises at least one radiation source 102, for instance at least one electroluminescent emitter, in this case an OLED. The radiation source 102 or each radiation source may be positioned in or on a mask, goggles or visor, or other such support structure or mount so as to be placed in a predetermined position relative to a patient's eye (or other area to be treated). The support structure may be for example as generally shown in FIGS. 1 and 2 and as described in WO2011/135362, and so will not be further described here. The apparatus further comprises a processor 104 (as a control element) and a battery 106 (or other source of power, for instance a power supply socket allowing a power supply wire to be coupled to the apparatus 100). The battery 106 is coupled to the processor 104 and the radiation source 102 so as to enable the supply of power to both.

The processor 104 is coupled to the radiation source 102 so as to control the operation of the radiation source. The apparatus further comprises a memory 108 coupled to the processor 104. The memory 108 is arranged to store instructions for controlling the processor 104 and data relating to the treatment regime, for instance intensity, waveform, frequency or pulse modulation of electromagnetic radiation emitted by the radiation source 102.

As used herein, the term ‘intensity’ is used to describe the luminance of a radiation source, that is the luminous intensity per unit area of light travelling in a given direction (measured by candela per metre squared—cd/m²).

As used herein, the term ‘pulse modulation’ is used to describe the duty cycle, pulse duration or pulse amplitude of emitted radiation.

The processor 104 is coupled to the radiation source 102 so as to control the operation of the radiation source, for example to turn the radiation source 102 on and off in accordance with a prescribed treatment regime.

FIG. 3 further illustrates a clock circuit 112 coupled to the processor 104. The clock circuit 112 is arranged to provide a timing signal allowing the processor 104 to calculate the times at which the apparatus is on or off, or the duration for which the apparatus 100 has been worn, or both. Data may be stored in memory 108.

As mentioned above, the memory 108 is arranged to store instructions for controlling the processor 104 and data relating to the treatment regime, for instance intensity, waveform, frequency or pulse modulation of electromagnetic radiation emitted by the radiation source 102. More specifically, the controller may be configured to vary the intensity, waveform, frequency or pulse modulation of radiation emitted from the apparatus over a predetermined program in accordance with the instructions from the memory. The apparatus may be one of several apparatuses available to be selected by a user (patient) or by a clinician such as an ophthalmologist or doctor. The available apparatuses may each have a different predetermined program relating to a particular treatment regime. Each treatment regime may have been set to suit particular diseases, patient gender, patient age ranges, or other parameter, or indeed a combination of two or more parameters.

In addition, one or more of the programs available may include an initial increase in radiation intensity, followed by a further period for treatment, and finally a decrease in intensity. For example, for many people, after they go to bed, the approximate first 30 minutes to 1 hour is the time period when they fall asleep and reach dark adaptation. Therefore, a gradual increase in radiation intensity will help allow the person to go to sleep, because the intensity is relatively low whilst they are somewhat awake, and the point of highest intensity is reached around the time they are fully asleep. In a similar matter, a gradual decrease in intensity over the 30 minutes to 1 hour prior to a person waking may help to minimise disturbance as the person is changing from the deepest sleep mode to waking.

The way in which the radiation intensity may be varied is, in this case, by control of the current that is fed to the OLED 102 from the battery 106, by the processor 104. Of course the intensity may be varied in other ways, e.g. controlling voltage for example.

Alternatively, or in addition, data may be passed from an input terminal 114 to the processor 104 for programming the dosage regime. For instance, a radio frequency (RF) receiver/antenna arranged to receive data wirelessly may be provided as the input terminal 114. The method of sending data between devices may be known as machine-to-machine monitoring (M2M). M2M monitoring may use short range wireless communication (for example in homes and hospitals) which may be via personal area networks (e.g. Bluetooth™, Zigbee™, MyWi™), or local area networks (e.g. Wi-Fi), or Near Field Communication (NFC). In a first example, the input circuit 114 may be an RFID reader such that data may be captured periodically. In a second example, input circuit 114 may comprise a Bluetooth® receiver arranged to receive data from an associated device such as a computer. In a third example, the input circuit 114 may comprise a NFC sensor that allows device to device communication, for example mask to mobile. It may act as a hub, e.g. coordinating with patient glucose levels. It could be self-powered or induction powered from the apparatus. It will be appreciated that further variations are possible, for instance using a wired connection to the apparatus. Wired connections may, for example, be via USB, FireWire™, Thunderbolt™ or Lightning™. Alternatively, data may be communicated via “Li-Fi” (the transmission of communication using visible light).

In this regard, as shown in FIG. 4, a system may include a patient monitoring device 200 and an apparatus 100. The apparatus 100 may include a radiation source 102, a processor 104 and an input terminal 114 (similarly to the arrangement shown in FIG. 3, for example). The patient monitoring device 200 may include a reader (or sensor) 202 for gathering data relating to the user, a processor 204 for processing the gathered data, and an output terminal 206 for transmitting the data to the input terminal 114 of the apparatus 100. The sensor may be a motion sensor, a thermal sensor or a sleep sensor, for example. The patient monitor 200 may optionally further include a memory 208 in which to store data before the data is transmitted to the apparatus 100.

The patient monitoring device may continually measure certain parameters associated with the patient and may transmit some or all of the data to the input terminal 114 of apparatus 100. The data may be transmitted continuously in real time, or optionally may transmit the data at periodic time intervals. The device 200 may selectively choose data to be transmitted, for example transmitting every one in 10 data readings, or other selection of the total readings. Patient parameters to be measured could include, for example, heart rate, blood pressure, blood flow, temperature, haemoglobin saturation (through the use of pulse oximetry), respiratory rate and eye movement. Parameters chosen may be parameters that are indicative of a particular state of the patient.

For example, in this embodiment, a patient's heart rate is measured by the patient monitoring device 200, which takes the form of a device including a sensor for monitoring the ECG, known per se in the art. Heart rate may be indicative of a patient's state of sleep, with a relatively slower rate indicating a deeper sleep and a relatively faster rate indicating a shallower sleep or state of non-sleep. In turn, heart rate data indicative of sleep state may be transmitted and used by the processor 104 to flag the intensity level of radiation to be emitted to the patient, for example with a higher intensity of radiation emitted (towards the patient's eye) during a time period when the heart rate is slower. As such, the system may actively control and vary the intensity of radiation emitted in response to patient data.

Some patient parameters that may be measured, for example glucose levels (a test requiring a droplet of blood), cannot be monitored continuously. In this case a patient monitoring device 200 may measure patient parameters periodically at a given time. The data from the patient monitor 200 may be transmitted directly to the apparatus 100 or optionally may be stored in memory 208 and transmitted at a later time.

Alternatively, as shown in FIG. 5, a system may include apparatus 100 and an external controller 300. In this case, the external controller is a computer. The external controller 300 includes a memory 308 for storing a database of information relating to different patient types and corresponding treatment regime programs (i.e. treatment regimes that have been found effective for particular patient types, e.g. for different genders, age ranges, diseases, and so on).

The memory 308 interacts with a processor 304. The processor 304 identifies the patient type information (for example via data received from a user interface (not shown) or from the memory itself), and interacts with the database of information held in the memory to identify the most appropriate treatment program to be given. The processor then sends the treatment program information to an output terminal 306 for sending the data to the input terminal 114 of the apparatus 100.

Alternatively, the functions performed by the external controller 300 may be performed by an ophthalmologist, doctor or clinician by reviewing the patient's medical details and determining an appropriate treatment program for the patient. Instruction data relating to a chosen treatment program may be input to the apparatus 100 via use of an external device or computer, e.g. similar to controller 300, or via a direct user input on the apparatus 100.

A yet further alternative system is shown in FIG. 6, in which a patient treatment apparatus 100 is linked to both a patient monitoring device 200 and an external controller 300. Such an arrangement essentially provides alternative options from which data or instructions can be sent to the apparatus 100. Data or instructions may be transmitted from the patient monitoring device 200, or from the external controller 300, or from both the patient monitoring device and the external controller. In the case of data or instructions from both, data must be assimilated and provided as a single set of instructions for the processor 104 of the apparatus 100. This assimilation of data may occur within the apparatus 100, or may occur at the external controller 300.

With this arrangement, a set of data specific to a patient using the apparatus 100 may be logged in the memory 308 and used to determine a future treatment program for that patient. More specifically, parameters that relate to patient health (e.g. heart rate, blood pressure, blood flow, temperature, haemoglobin saturation, respiratory rate, eye movement, etc. as mentioned before) are read by the patient monitoring device 200, sent to the output terminal 206 and then to the input terminal 302 of the external controller 300, logged in the memory 308 and assimilated with other data in the processor 304. Then the accumulated data may be used to generate an appropriate treatment program by the processor 304, before instruction data is sent to the apparatus 100 for controlling the radiation emitted by the apparatus 100.

In a similar manner, rather than general patient parameters such as those mentioned above, the system may be adapted to receive feedback from the patient about their recent sleep patterns when using the apparatus 100. This data may be used to tune the future program of radiation emission for that patient. For example, if the patient provides feedback to the external controller or doctor that their sleep pattern is hindered during a particular time period, then that information may be used to adjust the treatment program for the patient's ongoing use.

A method of manufacturing a patient treatment apparatus will now be described with reference to FIG. 7. In step S1 a radiation source is provided, for example an OLED, for providing electromagnetic radiation that can be directed towards a patient. In step S2 a mount element is provided, such as the facial mask mount shown in FIG. 1 or 2. The mount element is to be worn by a patient such that the mount aligns the radiation source with the area to be treated, in this case the eye. In step S3 a controller is provided, for example a processor 104 as described above. The controller may be provided on the mount element, or externally from the mount element. The controller is configured to vary the intensity, waveform, frequency or pulse modulation of radiation emitted from the radiation source in accordance with a treatment program that is based upon predetermined parameters relating to that patient.

Various other modifications to the detailed arrangements as described above are possible. Although the radiation source has been described above as an OLED, this may be any electroluminescent emitters, light emitting device, light emitting cell (LEC), light emitting electrochemical cell (LEEC), LED or similar devices.

For example, the apparatus 100 may optionally include an integrated alarm. The alarm may be set to wake up the patient at a predetermined wake up time. The alarm may be in the form of a sound (for example a buzz, a ring or a chime) and/or may signal to the processor 104 to increase the intensity of the radiation source 102 (providing a sunrise imitation) to thereby wake the patient gradually.

The apparatus 100 may optionally include a further alarm system which may be connected to the input terminal 114 (or a further input terminal) or the processor 104. The memory 108 may have stored ideal or safe patient parameter ranges. If the input terminal 114 receives patient parameter information (from the patient monitoring device 200 or other suitable means) which is outside of the ideal or safe range, the alarm system will trigger. The alarm system may, for example, be in the form of a sound emitting form the apparatus 100, or may alert other parties (for example a health professional, family member, or neighbour) through wireless communication. This arrangement provides the advantage that if a user becomes unwell, a suitable person will be notified and can subsequently take appropriate action to help the user.

The processor 104 may optionally be further arranged to control the wavelength of electromagnetic radiation emitted. The wavelength may be actively controlled throughout the treatment regime according to predetermined parameters or according to data received from a patient monitoring device, for example. In this arrangement the radiation source may be a stack of LEDs, OLEDs, LECs or LEECs, for example, arranged in a suitable way such that the required range of wavelengths are obtainable. Aptly, the wavelengths obtainable are between 460 and 550 nm when treating diabetic retinopathy or wet AMD or birdshot chorioretinopathy, or 650 to 690 when treating dry AMD, for example. Multiple approaches may be taken to producing a variable wavelength (multicolour) device, from the use of a stacked OLED in which using transparent electrodes independent devices can be placed on top of one another in the manufacturing process. Alternatively the OLEDs could be pixelated, with adjacent pixels having different colours that can be lit independently. A third possibility is the use of integrated or separately applied photonic structures such as Bragg Reflectors covering the device, which selectively allows or reflects particular wavelengths of light incident upon them, and thus can be used to narrow the emission bandwidth of a device.

With the above-described arrangements the intensity and/or optionally also the wavelength, waveform, frequency or pulse modulation of electromagnetic radiation used to treat an area of a patient may be varied in accordance with predetermined parameters, for example parameters associated with the patient themselves, or parameters associated with generalisations of the type of patient being treated. The parameters may include temperature, blood pressure level, the patient's age gender or race, the patient's response to test radiation levels, for example.

The treatment regime applied to a patient may therefore be specifically chosen from a database of information known about certain patient types (age ranges, race, gender etc.), or may be chosen based on feedback from the patient themselves, or direct readings taken from the patient themselves.

Furthermore, as the database of information stored in the memory increases with further use, the data will become more useful in determining treatment programs and identifying generalisations about patient types.

It will be clear to a person skilled in the art that features described in relation to any of the embodiments described above can be applicable interchangeably between the different embodiments. The embodiments described above are examples to illustrate various features of the invention.

Further embodiments of the present invention are described in the numbered paragraphs below.

1. A method of operating a medical apparatus for emitting radiation towards an area to be treated of a patient, comprising:
determining a radiation treatment program for a patient;
inputting instructions indicative of the treatment program into the medical apparatus; and
controlling a radiation source to emit electromagnetic radiation according to the instructions.

2. A method of assembly of an apparatus comprising
selecting a desired wavelength/intensity/waveform/pulse modulation of radiation; and
setting a radiation source in accordance with the desired wavelength/intensity/waveform/pulse modulation.

3. A method as described in paragraph 2, further comprising identifying the requirements of the user and selecting the desired wavelength in accordance with the identified requirements.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A medical apparatus comprising:
a radiation source for emitting electromagnetic radiation towards an area to be treated of a patient;
a mount element arranged to be worn by the patient for positioning the radiation source in a predetermined position relative to the area to be treated; and
a controller for controlling the duration or time that the radiation source emits electromagnetic radiation, and for varying the intensity, waveform, frequency or pulse modulation of electromagnetic radiation emitted in accordance with predetermined parameters.

2. A medical apparatus as claimed in claim 1, wherein the controller is adapted to vary the intensity, waveform, frequency or pulse modulation of radiation emitted in accordance with a treatment program corresponding to the predetermined parameters, the program received at an input terminal.

3. A medical apparatus as claimed in claim 2 wherein the input terminal is a user interface provided on the apparatus, or a receiver element arranged to receive a signal from a remote device.

4. A medical apparatus as claimed in claim 2 wherein the treatment program comprises an increase in intensity and a decrease in intensity.

5. A medical apparatus as claimed in claim 1 wherein the controller is provided remotely to the mount element.

6. A medical apparatus as claimed in claim 1 wherein the treatment program is predetermined based on a patient's personal needs.

7. A medical apparatus as claimed in claim 6 wherein the treatment program is predetermined based on a set of data previously logged from said patient.

8. A medical apparatus as claimed in claim 1 further comprising a clock device for providing a timing signal to the controller.

9. A medical apparatus as claimed in claim 8 further comprising an alarm for making an indication to the patient at a predetermined wake up time.

10. A medical apparatus as claimed in claim 1 wherein the controller gradually changes the intensity, waveform, frequency or pulse modulation of radiation emitted over the first period of treatment and gradually changes the intensity, waveform frequency or pulse modulation over the final period of treatment, wherein the first period of treatment and the final period of treatment are between about 30 minutes and 1 hour.

11. A medical apparatus as claimed in claim 2, wherein the input terminal, or a further input terminal, is configured for receiving patient data indicative of real time information associated with the patient and sending the patient data to the controller to actively control the radiation source.

12. A medical apparatus as claimed in claim 11 further comprising an alarm system connected to the input terminal or further input terminal for indicating when patient data has surpassed a predetermined boundary or boundaries.

13. A medical apparatus as claimed in claim 1, wherein the controller is further arranged to actively control the wavelength of electromagnetic radiation emitted.

14. A medical apparatus as claimed in claim 13, wherein the controller is arranged to control the intensity of the electromagnetic radiation emitted between 30 and 100 cd/m2.

15. A medical apparatus as claimed in claim 1, wherein the radiation source comprises at least one organic light emitting diode (OLED).

16. A system comprising:
a patient data monitoring apparatus for taking readings of a parameter associated with a patient; and
a treatment apparatus for being worn by the patient, comprising:
a radiation source for emitting electromagnetic radiation towards an area to be treated of a patient;
a mount element for positioning the radiation source in a predetermined position relative to the area to be treated; and
a controller for controlling the intensity, waveform, frequency or pulse modulation of electromagnetic radiation emitted,
wherein the treatment apparatus is arranged to receive patient data, indicative of the readings or a proportion of the readings, from the patient data monitoring apparatus, and wherein the controller is arranged to vary the intensity, waveform, frequency or pulse modulation of the electromagnetic radiation emitted in accordance with the patient data.

17. A system as claimed in claim 16, wherein the patient data monitoring apparatus is arranged to send data indicative of the readings or a proportion of the readings directly to a receiver on the treatment apparatus.

18. A system comprising:
a treatment apparatus for being worn by a patient, comprising
a radiation source for emitting electromagnetic radiation towards an area to be treated of a patient, and
a mount element for positioning the radiation source in a predetermined position relative to the area to be treated; and
a controller for generating signals to control and vary the intensity, waveform, frequency or pulse modulation of electromagnetic radiation emitted by the radiation source, in accordance with predetermined parameters.

19. A system as claimed in claim 18 wherein the controller is adapted to vary the intensity, waveform, frequency or pulse modulation of radiation emitted in accordance with a treatment program corresponding to the predetermined parameters.

20. A method of manufacturing a medical apparatus comprising:
providing a radiation source for emitting electromagnetic radiation;
providing a mount element to be worn by a patient for positioning the radiation source in a predetermined position; and
providing a controller for controlling the duration or time that the radiation source emits electromagnetic radiation, and varying the intensity, waveform, frequency or pulse modulation of electromagnetic radiation emitted in accordance with predetermined parameters.

21. (canceled)

22. (canceled)

23. (canceled)
